(19) United States
(12) Patent Application Publication
(10) Pub. No.: US 2018/0035927 A1
(43) Pub. Date: Feb. 8, 2018

(54) WEARABLE DEVICE FOR SWEAT TESTING ADMINISTRATION

(71) Applicant: KONINKLIJKE PHILIJPS N.V., EINDHOVEN (NL)

(72) Inventors: John Cronin, Bonita Springs, FL (US); Steven Philbin, Tuscaloosa, AL (US)

(21) Appl. No.: 15/552,403

(22) PCT Filed: Mar. 8, 2016
(86) PCT No.: PCT/EP2016/054836

§ 371 Date: Aug. 21, 2017

Related U.S. Application Data
(60) Provisional application No. 62/130,222, filed on Mar. 9, 2015.

(30) Foreign Application Priority Data
Jul. 16, 2015 (EP) 15177095.5

(51) Int. Cl. A61B 5/145; G06F 19/00; A61B 5/00

(52) U.S. Cl. CPC A61B 5/14517; A61B 5/4875; A61B 5/4806; A61B 5/14546; A61B 5/746; G06F 19/3406; G06F 19/3418; G06F 19/345; G06F 19/3481; A61B 5/681; A61B 5/11

(57) ABSTRACT

Various embodiments relate to a method of assisting a user with administering a sweat test based on monitoring one or more physiological conditions of the user by a wearable device. In an embodiment, a wearable device may receive an indication of a selected sweat-test type from the user and determine as a function of the sweat-test type, which of the plurality of physiological sensors to use for data collection. Following data collection, it may be determined on the wearable device, if one or more test-triggering physiological conditions are present in the user. If one or more test-triggering physiological conditions are present, instructions to deploy the sweat-test strip to a location of the user and to start the sweat test may be provided to the user. Instructions may also be provided to the user to end the sweat test.

### RELATED APPLICATION DATA

This application claims the benefit of priority of U.S. Provisional Patent Application Ser. No. 62/130,222, filed on Mar. 9, 2015, and titled “METHODS, SYSTEMS, AND SOFTWARE FOR PROVIDING PERSPIRATION HEALTH DATA TO A USER”, which is incorporated by reference herein in its entirety for all purposes.

### TECHNICAL FIELD

Various embodiments disclosed herein relate generally to the field of wearable technology. More specifically, but not exclusively, various embodiments are directed to computer implemented methods a machine readable storage medium for assisting a user with administering a sweat test using a sweat test strip and a wearable device comprising a plurality of physiological sensors.

### BACKGROUND

A sweat test may use various types of biosensor strips that rest or stick to the surface of the skin and measure chemicals that may be present in a user's sweat. In the race to develop highly useful and marketable sweat tests, various biosensor strips have been designed and configured to measure for electrolytes. Such a sweat test may allow a user to determine if they are dehydrated or hydrated.

### SUMMARY

Various embodiments disclosed herein are directed to a computer-implemented method of assisting a user with administering a sweat test using a sweat test strip and a wearable device comprising a plurality of physiological sensors. The method includes receiving, via the wearable device, an indication of a selected sweat-test type from the user, determining, on the wearable device and based on the selected sweat-test type, which one or more of the plurality of physiological sensors to use for sensing one or more test-triggering physiological conditions of the user, collecting, on the wearable device, sensor data using the determined one or more of the plurality of physiological sensors, determining, on the wearable device using the sensor data, whether or not the one or more test-triggering physiological conditions are present in the user, in response to determining that at least one of the one or more test-triggering physiological conditions is present, instructing, by the wearable device, the user to deploy the sweat test strip to a location on the user and to initiate the sweat test; and instructing, by the wearable device, the user to end the sweat test.

Various embodiments disclosed herein are directed to a machine-readable storage medium containing machine-executable instructions for assisting a user with administering a sweat test using a sweat test strip and a wearable device comprising a plurality of physiological sensors. The machine-executable instructions include a first set of machine-executable instructions for receiving, via the wearable device, an indication of a selected sweat-test type from the user; a second set of machine-executable instructions for determining, on the wearable device and based on the selected sweat-test type, which one or more of the plurality of physiological sensors to use for sensing one or more test-triggering physiological conditions of the user; a third set of machine-executable instructions for collecting, on the wearable device, sensor data using the determined one or more of the plurality of physiological sensors; a fourth set of machine-executable instructions for determining, on the wearable device using the sensor data, whether or not the one or more test-triggering physiological conditions are present in the user; a fifth set of machine-executable instructions for, in response to determining that at least one of the one or more test-triggering physiological conditions is present, instructing, by the wearable device, the user to deploy the sweat test strip to a location on the user and to initiate the sweat test; and a sixth set of machine-executable instructions for instructing, by the wearable device, the user to end the sweat test.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 illustrates an example of a flow diagram illustrating a method for assisting a user with administering a sweat test using a sweat test strip and a wearable device comprising a plurality of physiological sensors;

FIG. 2 illustrates an example of a sweat data matrix;

FIG. 3 illustrates an example diagrammatic representation of a wearable sweat analysis (WSA) system in accordance with an embodiment of the invention;

FIG. 4 illustrates an example diagrammatic representation of an exemplary wearable computing device that can be used to implement one or more features of the present invention;

FIG. 5A illustrates an example of a sweat test strip in accordance with an embodiment of the invention;

FIG. 5B illustrates the sweat test strip of FIG. 5A placed on the forearm of a user;

FIG. 6 illustrates an exemplary user start graphical user interface (GUI) in accordance with an embodiment;

FIG. 7A illustrates an exemplary user alert GUI in accordance with an embodiment;

FIG. 7B illustrates an exemplary sweat analysis sensor data GUI in accordance with an embodiment;

FIG. 8 illustrates an exemplary network alert database;

FIG. 9A illustrates an exemplary network identification database;

FIG. 9B illustrates an exemplary wearable alert database;

FIG. 10A illustrates an exemplary wearable sensor database;

FIG. 10B illustrates an exemplary wearable sweat analysis sensor database;

FIG. 11 is a flow diagram illustrating a wearable software method;

FIG. 12 is a flow diagram illustrating a wearable identification software method and a network identification software method;

FIG. 13 is a flow diagram illustrating a wearable manual choice software method;

FIG. 14A is a flow diagram illustrating a wearable monitoring software method;

FIG. 14B is a flow diagram illustrating a wearable action software method;

FIG. 15 illustrates particular implementations of various steps of a method for providing sweat health data to a user; and

FIG. 16 illustrates a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted from the figures.

### DETAILED DESCRIPTION

The description and drawings presented herein illustrate various principles. It will be appreciated that those skilled in the art will be able to devise various arrangements that, although not explicitly described or shown herein, embody these principles and are included within the scope of this disclosure. As used herein, the term, “or,” as used herein, refers to a non-exclusive or (i.e., and/or), unless otherwise indicated (e.g., “or else” or “or in the alternative”). Additionally, the various embodiments described herein are not necessarily mutually exclusive and may be combined to produce additional embodiments that incorporate the principles described herein.

As with most burgeoning technologies, sweat tests and related systems have not yet been optimized with regard to cost. Accordingly, it would be desirable to provide improved sweat tests that use biosensor strips at a lower cost generally. Various additional benefits of the system described herein will be apparent in view of the present description.

Aspects of the present disclosure are directed to wearable technology devices, for example, smartwatches, health-bands, fitness-bands, and smartphones, among others, and combinations thereof, that are enabled to assist their wearers in preventing and/or treating physiological conditions, such as dehydration, stress, fatigue, muscle fatigue, infection, and/or depression by instructing a user when to take a sweat test. Thus, wearable devices include attachable wearable devices that are attached to the body (e.g., wrist, neck, ankle, waist, ear, etc.) as well as carried devices (e.g., mobile phones). Accordingly, various embodiments described herein are adapted to monitor and decide when a person should test their sweat in accordance with the occurrence of one or more certain physiological conditions. Because some physiological conditions can be highly debilitating and even life-threatening, enabling a wearable device to provide such functionality can allow users of this technology to not only quickly recognize certain physiological states, but also, for example, to take remedial measures as directed by the wearable device. Furthermore, by providing a user with an indication of when to take a sweat test eliminates guesswork that may cost the user money. For example, taking a sweat test when prompted may prevent unnecessary sweat tests and costs associated therewith. As described below in detail, such aspects may be facilitated by various GUI's, and other software features running on one or more of a variety of devices, including wearable technology devices (or simply “wearable devices”), and web servers, among other devices. These broad aspects of the present invention are described below in connection with a variety of specific examples. That said, those skilled in the art will readily understand that the specific examples described are just that: examples that will inform and instruct those skilled in the art about broad features that they can then implement in a plethora of ways using only routine knowledge and skill in the art.

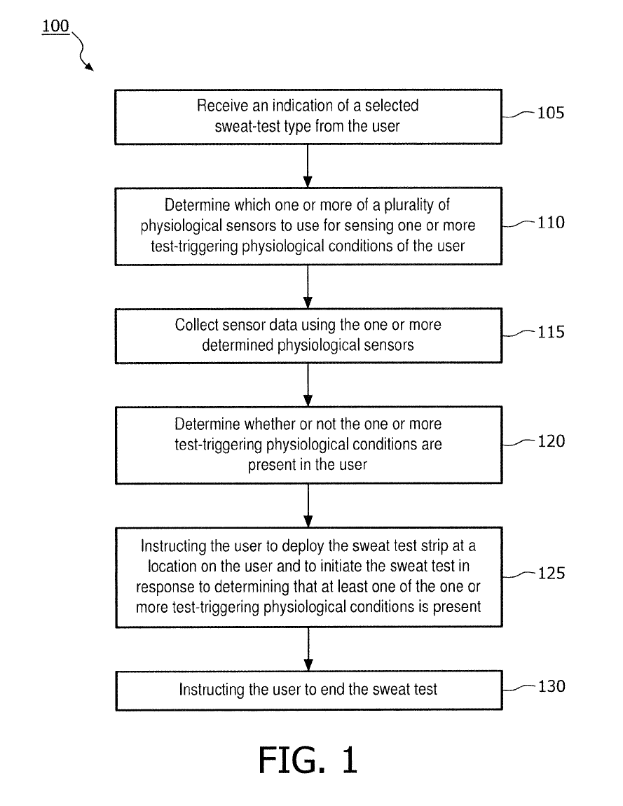

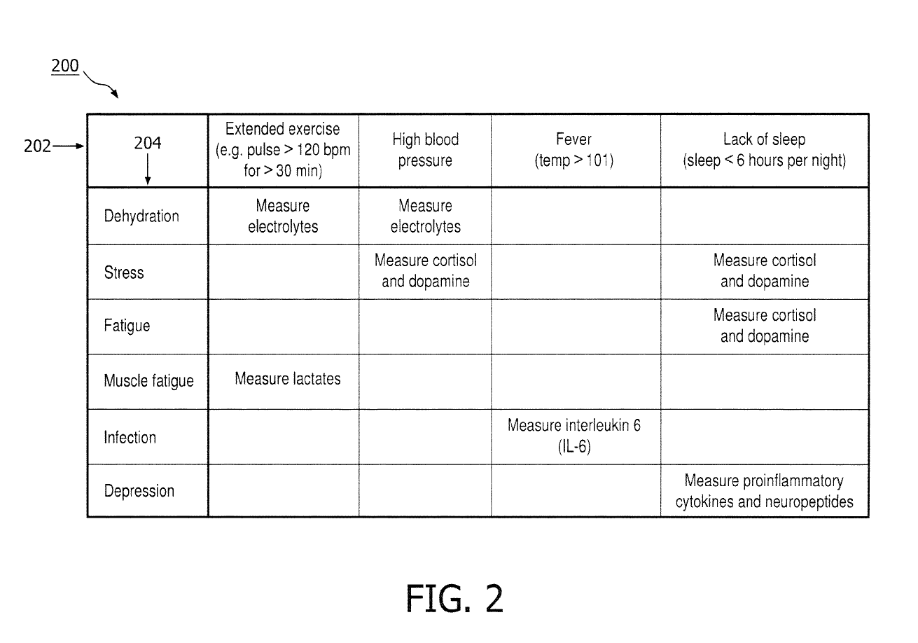

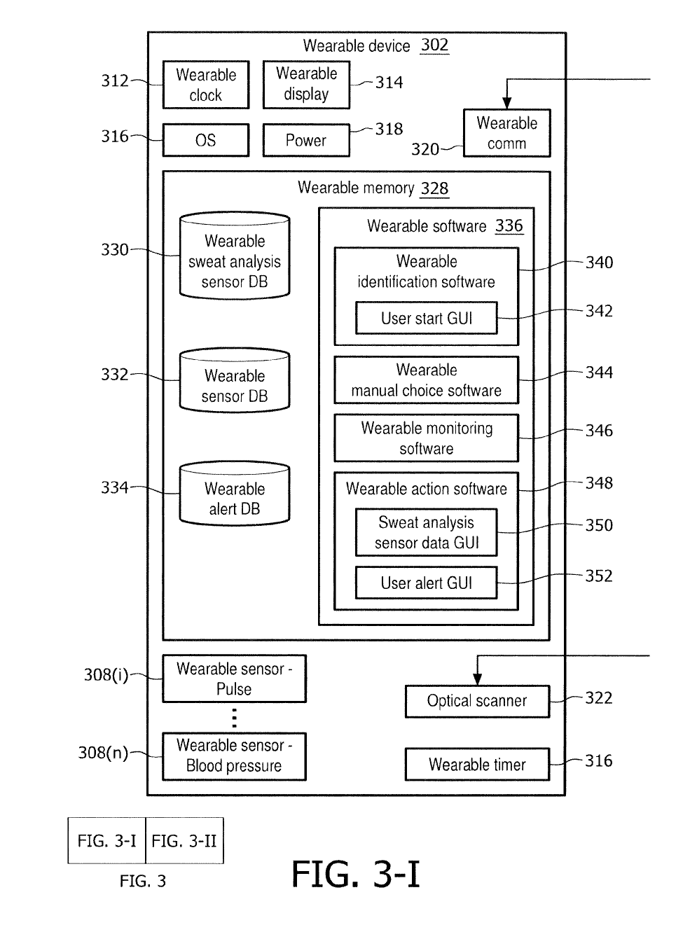

Turning now to the drawings, FIG. 1 illustrates an exemplary overall method 100 that can be performed by wearable software executed on one or more devices of a WSA, such as exemplary WSA system 300 of FIG. 3. Before describing overall method 100 of FIG. 1, a sweat data matrix 200 of FIG. 2 and a WSA system 300 of FIG. 3 are first described to give the reader an example context in which the overall method 100 may be executed.

Referring now to FIG. 2, in the embodiment shown, sweat data matrix 200 includes a row 202 containing test-triggering physiological conditions that is crossed with a column 204 containing health conditions. It will be apparent that various alternative data structures may be used to store and use the information (or similar types of information) illustrated in FIG. 2. For example, Each health condition in the column 204 may be associated with a rule table, including multiple rules each of which corresponds to one of the physiological conditions in the row 202 and corresponding measurement recommendation.

Test-triggering physiological conditions, such as those in row 202, may include but are not limited to extended exercise, high blood pressure, fever, and/or lack of sleep. In various embodiments, such physiological conditions may be identified in the wearer by analyzing current, recent, or historical sensor data from one or more wearable devices (such as, in some embodiments, a wearable device storing or analyzing the matrix 200). Health conditions, such as the health conditions provided in column 204, may include but are not limited to dehydration, stress, fatigue, muscle fatigue, infection, and depression. In various embodiments, these health conditions may be manually-identified by the wearer, a physician, or caregiver as being exhibited by the user or as being relevant for tracking for the user. Alternatively, in some embodiments, sensor data for the user may be used in conjunction with, e.g., a trained model (e.g., trained according to logistic regression, neural networks, or other machine learning approached) to draw conclusions about health conditions exhibited by the user. In some embodiments, only those rows 204 (or corresponding structures, e.g., rule tables) associated with a health condition exhibited by the wearer or identified as relevant for tracking for the user may be loaded (e.g., in the wearable device) for evaluation.

Sweat data matrix 200 provides examples of test-triggering physiological conditions that may indicate if a sweat test, such as a sweat-test type to measure electrolytes of a user, should be taken and/or the health condition, such as dehydration, that may be monitored by taking the sweat test. Extended exercise may be indicated by a user pulse of greater than 120 beats per minute for greater than 30 minutes and may be cause to measure electrolytes to monitor dehydration. Extended exercise may also be cause to measure lactates to monitor muscle fatigue. In another example, high blood pressure may be cause to measure electrolytes to monitor dehydration and/or to measure cortisol and dopamine to monitor stress. In yet another example, fever, such as a temperature greater than 101° F. (38.3° C.), may be cause to measure interleukin six (IL-6) to monitor infection. In another example, lack of sleep, such as sleeping less than 6 hours per night, may be cause to measure cortisol and dopamine to monitor stress and/or fatigue or to measure Proinflammatory cytokines and neuropeptides to monitor depression. Sweat data matrix 200 is provided as an example only and the test-triggering physiological conditions of row 202 and the health conditions of column 204 are not limited to those shown. In addition, sweat-test types are not limited to those shown here. After reading this disclosure in its entirety, a person of ordinary skill in the wearable technology art will appreciate that any appropriate number of test-triggering physiological conditions may indicate sweat-test types that may be capable of monitoring a variety of different health conditions.

Referring now to FIG. 3, an example of a wearable sweat analysis (WSA) system 300 includes a wearable device 302 and a server 304 that communicate with one another via one or more communications networks 306 such as, for example, a local area network, carrier network, cloud computing environment network, or the Internet. While the server 304 is identified as a singular server in various examples, in other embodiments the various functions described herein as being performed by the server 304 may be distributed among multiple separate servers (e.g., a set of servers forming a service provider network) such as multiple virtual machines in a cloud computing environment. While not shown, those skilled in the art will readily understand that various specific communications systems will be present, such as, for example, wireless data communications systems (e.g., cellular-based communications systems and satellite-based communications systems, WI-FED communications systems, etc.) and wired communications system (e.g., optical fiber based communications systems, copper wire based communications systems, etc.). Such systems may work together to provide the point-to-point communications needed between wearable device 302 and server 304. Wearable device 302 may be worn by a user on any appropriate part of their body. For example, wearable device 302 may be worn on a wrist, forearm, upper arm, leg, waist, neck, ear, and/or back and may include physiological sensors, such as physiological sensors 308(1) to 308(N) dimensioned and configured for data collection at specific locations on the body. In addition, physiological sensors 308(1) to 308(N) may be in contact with the user. Wearable device 302 may be worn by a person or another entity, and may record data related to the individual and/or the individual's surroundings.

Still referring to FIG. 3, WSA system 300 may also include at least a sweat test strip 310. Sweat test strip 310 may further include a strip ID barcode 312 (or other indicia such as a QR code or textual code capable of optical character recognition). Wearable device 302 includes a wearable clock 312, a wearable display 314, a wearable timer 316, an operating system (OS) 316, a power 318, such as a battery or other appropriate power source, a wearable communication interface 320, shown here as “Wearable Comm”, that may use e.g., BLUETOOTH™ and/or WI-FI™, and an optical scanner 322 (e.g. a camera) that may be used to read strip ID barcode 312 on sweat test strip 310. Strip ID barcode 312 may be referred to herein interchangeably with strip identification barcode. As noted above, wearable device 302 may also include one or more wearable sensors 308(1) to 308(N) such as, for example, a pulse sensor 308(1) and blood pressure sensor 308(N). Herein, pulse sensor 308(1) and blood pressure sensor 308(N) may be referred to as “physiological sensors.” It is noted that the plurality of physiological sensors 308(1) to 308(N) may include any appropriate number of physiological sensors onboard wearable device 302 and the physiological sensors may not be limited to monitoring a user's pulse and blood pressure. For example, various ones of physiological sensors 308(1) to 308(N), if provided, may be designed and configured to monitor sleep or temperature. It will be apparent that, in some embodiments, such sensors 308(1-N) may be used in conjunction with interpretation instructions (not shown) in the memory 328 for execution by the processor (not shown) to extract parameters from the raw data gathered by the hardware sensors 308(1-N). For example, the pulse sensor 308(1) may include an optical sensor for capturing a color or other value of the user's skin underlying the sensor; software executed by the processor (not shown) may then process this raw data (e.g., the variations in color over time) to estimate a user's pulse.

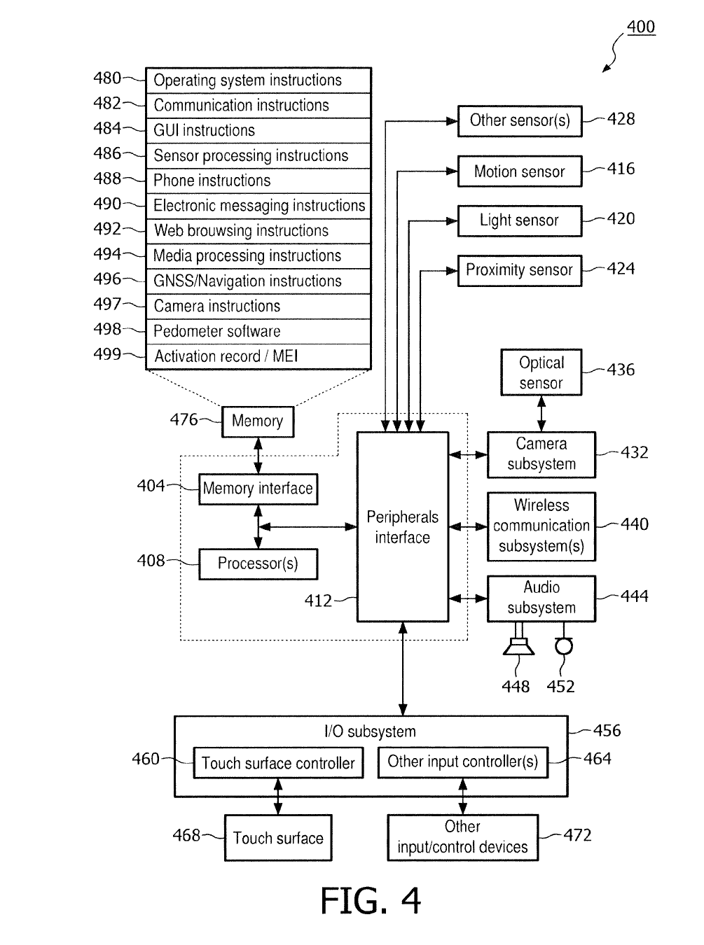

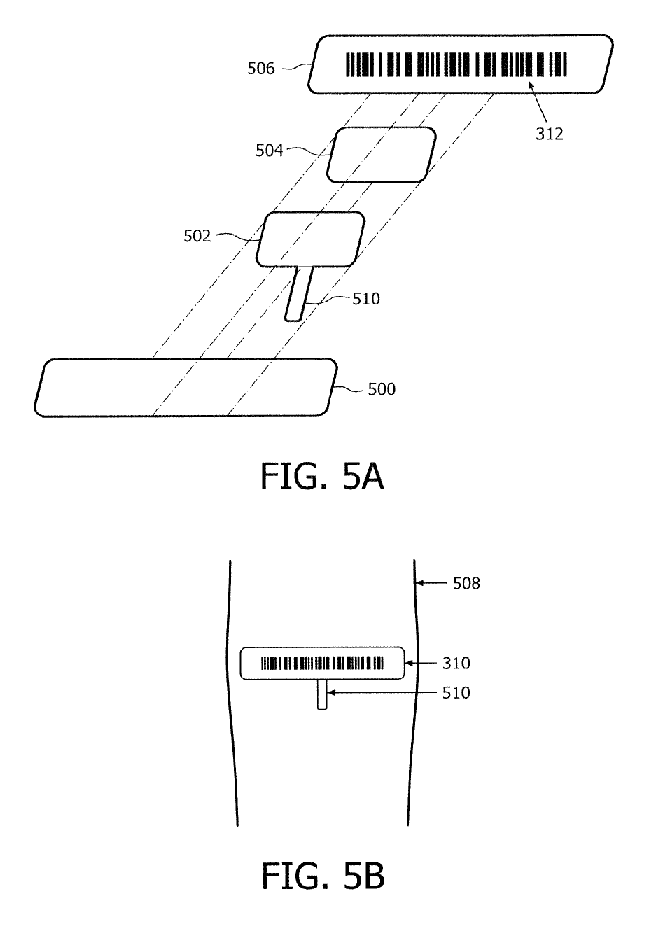

In some embodiments, the sweat test strip may be held by the wearable device 302 prior to and during use. In particular, the body of the wearable device 302 may include a slot or other cavity for holding the sweat test strip 310 against the body of the user. In some embodiments, such as those described with regard to FIGS. 5A-B, the sweat test strip may include a pulltab or other structure for enabling the user to activate the test strip (e.g., by allowing sweat to come into contact with a testing pad). In some such embodiments, the body of the wearable device may include an aperture for allowing the activation structure to extend from the body of the wearable device such that the activation structure may be accessed by the user. In other embodiments, the wearable device may include mechanical components (specific examples of which will be apparent to those of skill in the art) for removing a protective cellophane layer or for otherwise activating a sweat test strip. Upon use of the test strip, the user may be able to remove and dispose of the test strip and insert a new, unused test strip into the wearable device (e.g., by sliding the test strips through a slot or by opening a compartment, or removing a frame structure). In such embodiments, variations to the example test strip of FIGS. 5A-B will be apparent; for example, in some such embodiments the test strip need not be provided with an adhesive layer because the wearable device may sufficiently hold the strip against the skin of the user.

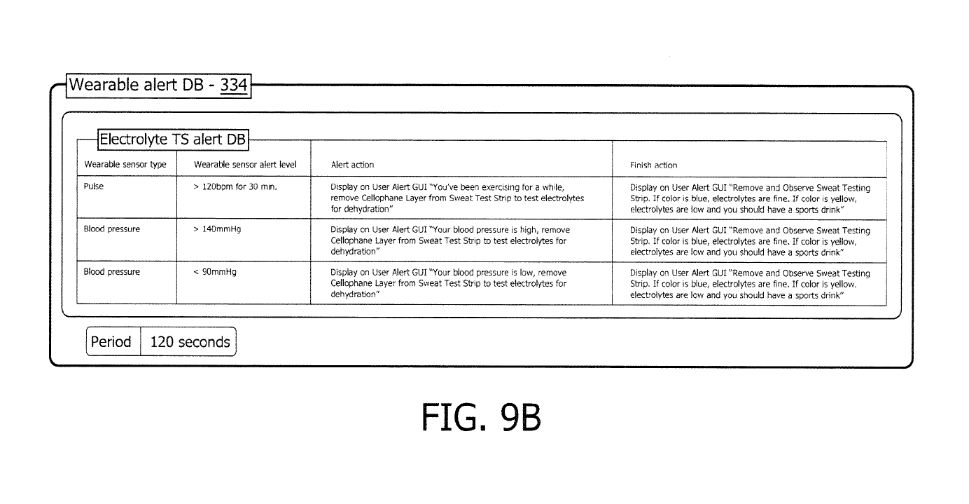

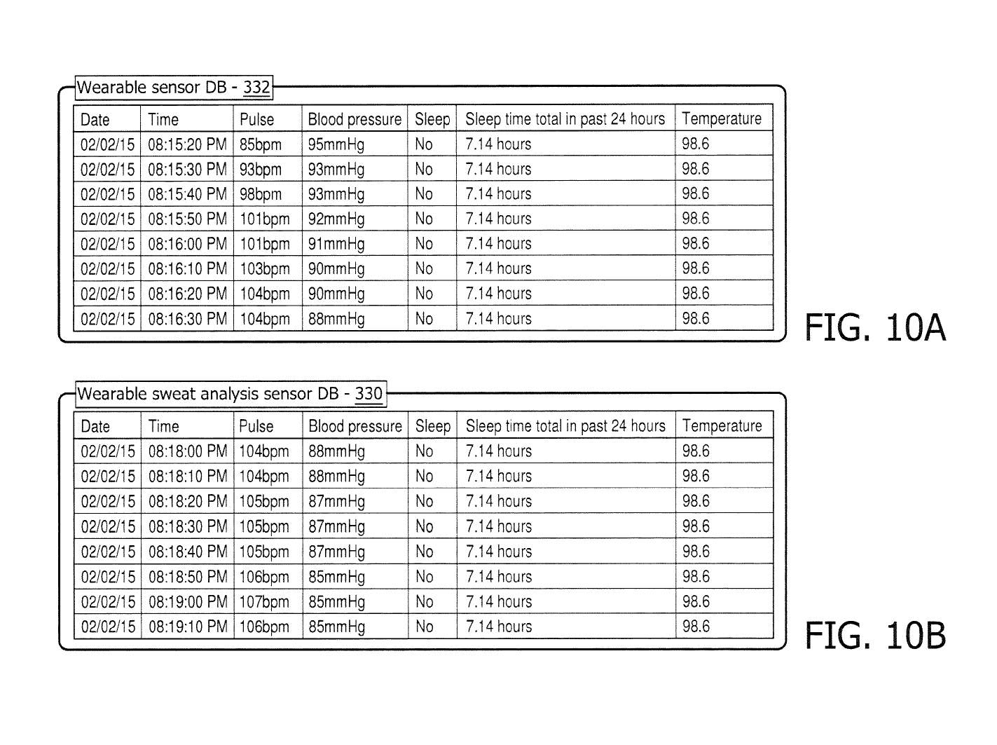

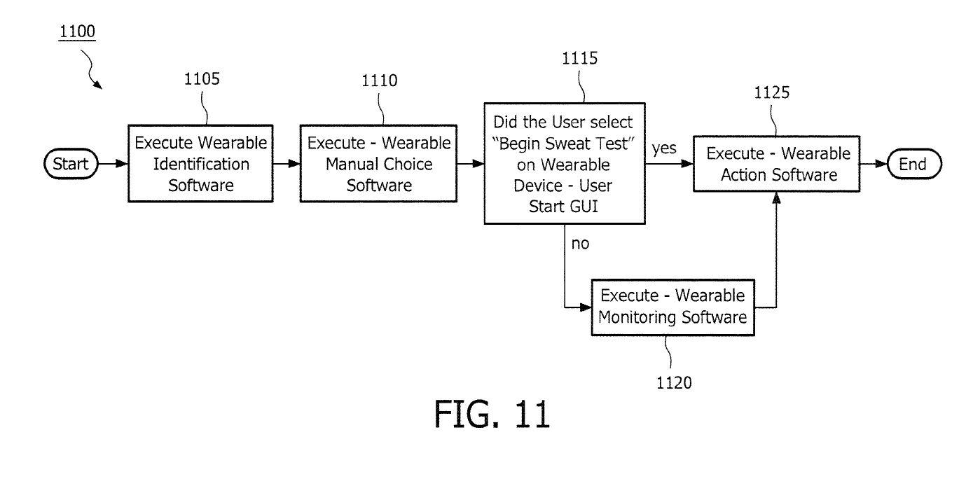

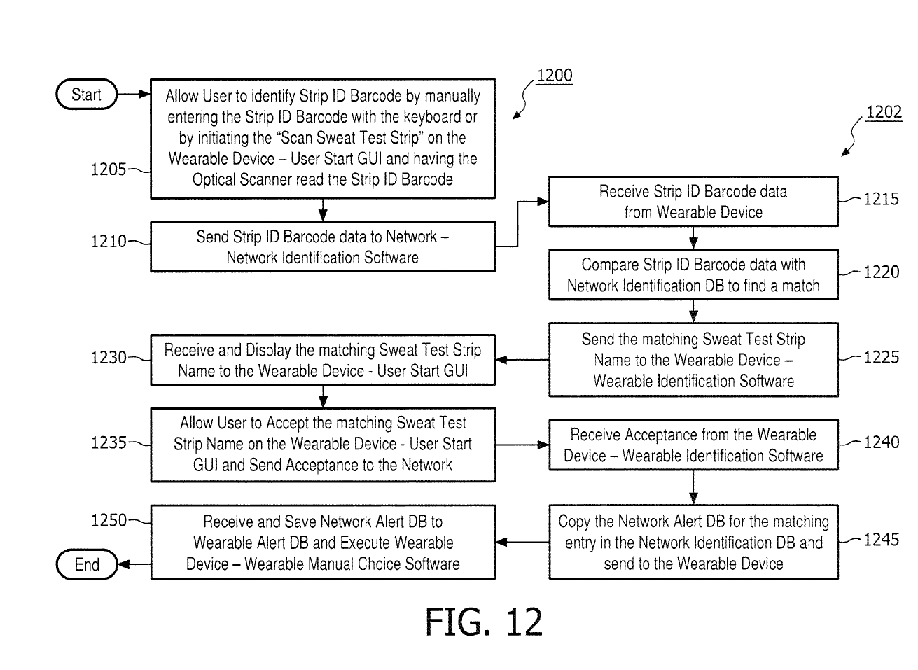

Wearable device 302 may further include a wearable memory 328 that contains a wearable sweat analysis sensor database 330, a wearable sensor database 332, and a wearable alert database 334. Wearable sweat analysis database 330 may be used to store sensor data collected by one or more of physiological sensors 308(1) to 308(N) during a sweat test. Wearable sensor database 332 may be used to store sensor data collected by one or more of physiological sensors 308(1) to 308(N) when a sweat test is not being conducted. Wearable alert database 334 may be used to store test-triggering physiological conditions, instructions to deploy sweat test strip 310 to a location on the user, and instructions to end the sweat test. Further discussion of wearable alert database 334, wearable sensor database 332, and wearable sweat analysis sensor database 330 is provided herein in the context of FIGS. 9B to 10B, respectively.

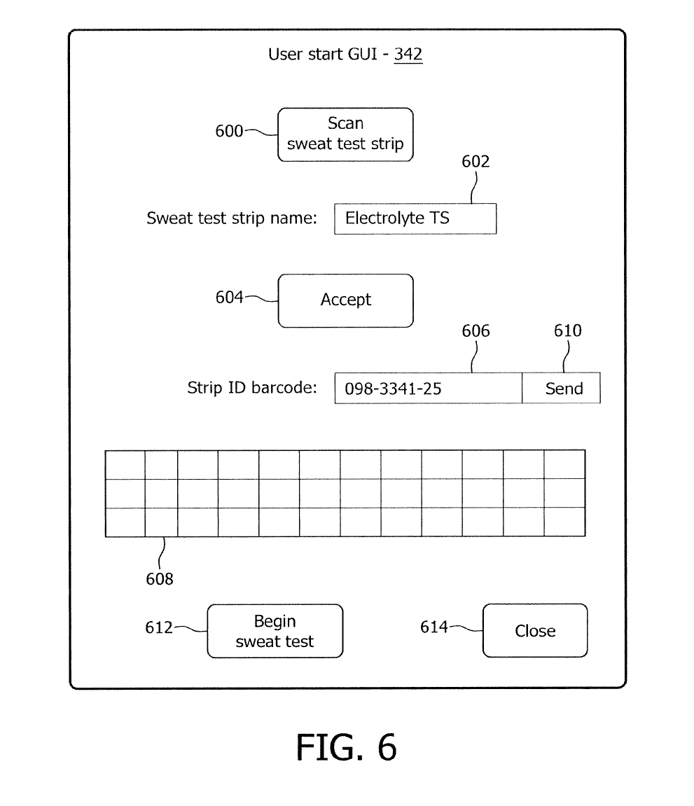

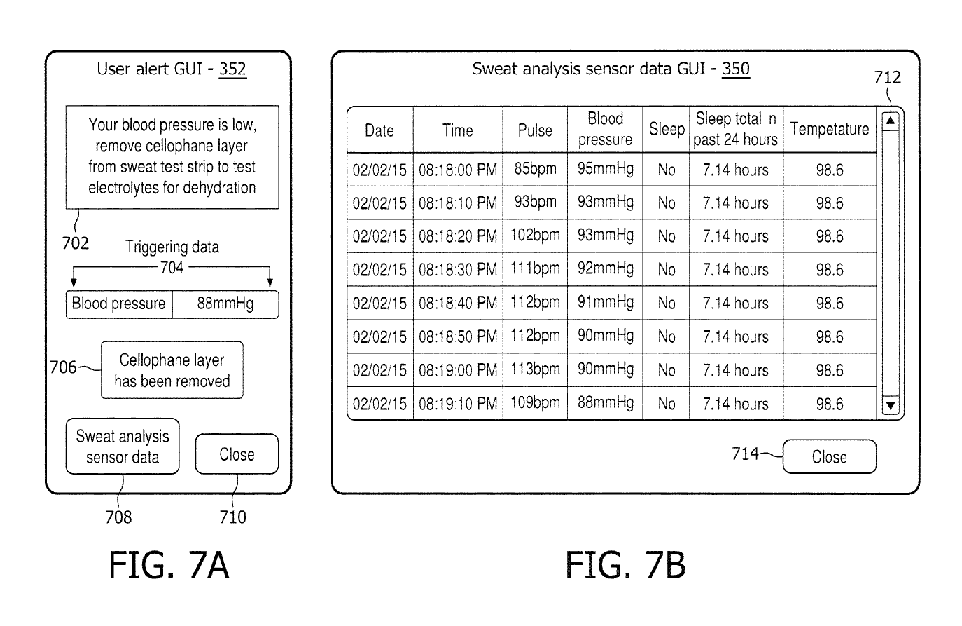

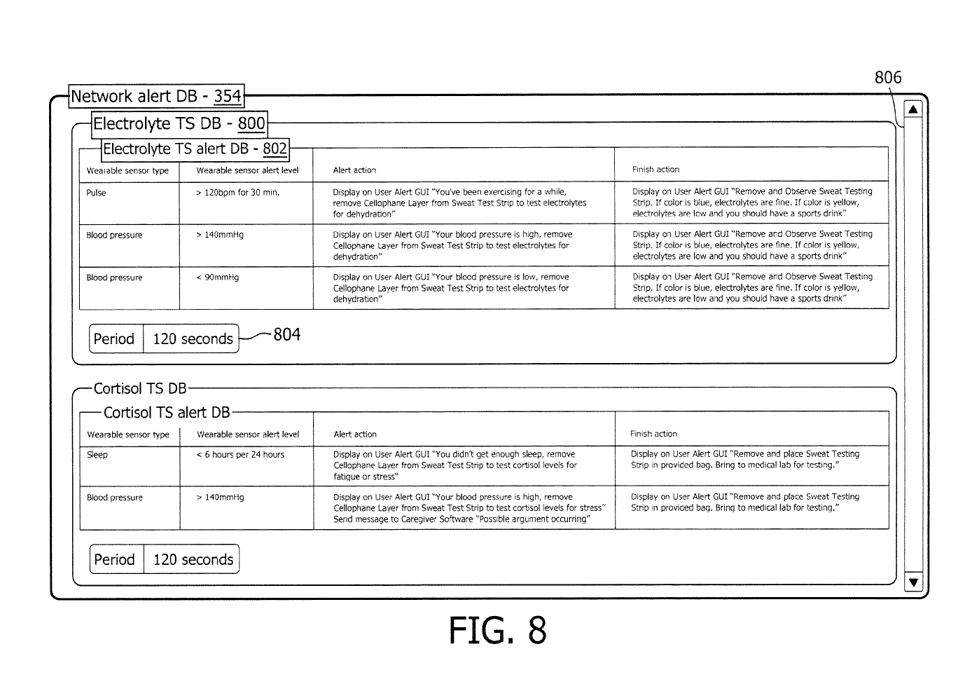

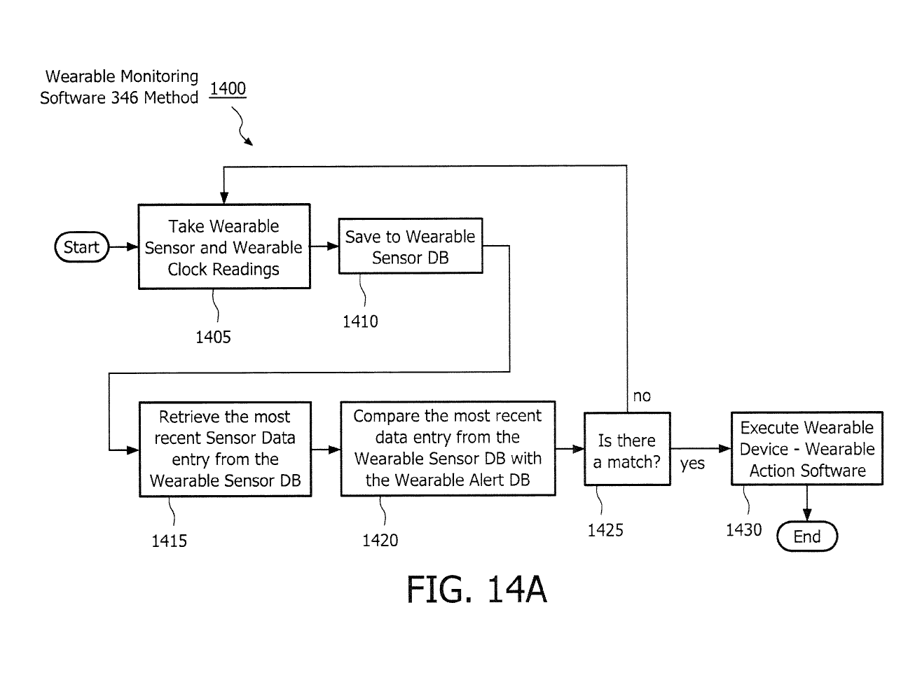

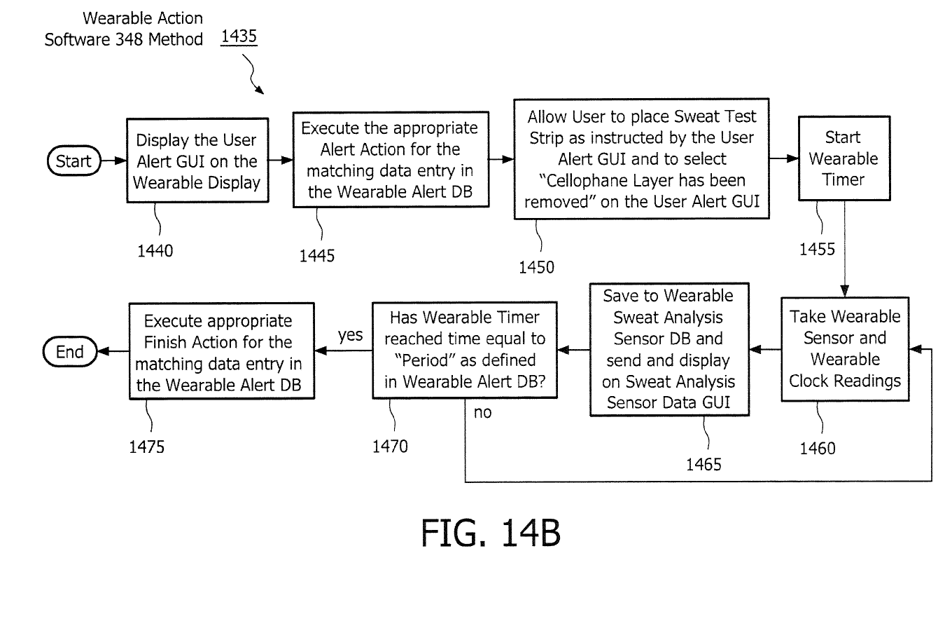

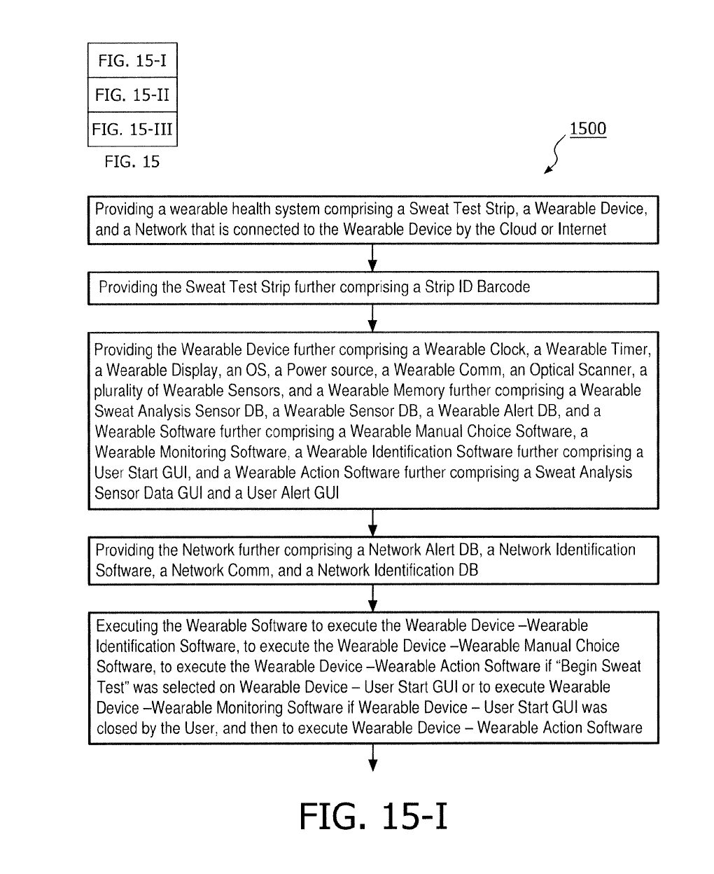

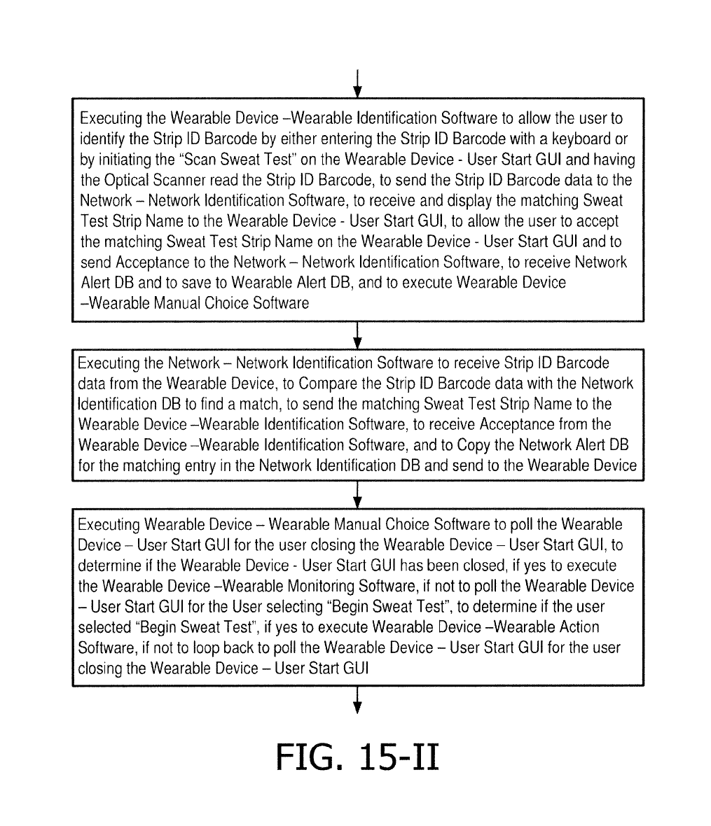

Wearable device 302 may further include wearable software 336, which may comprise wearable identification software 340 as described further herein in the context of FIG. 12. Wearable software 336 may control the overall operation of WSA system 300. Wearable identification software 340 may also include a user start GUI 342, as described further herein in the context of FIG. 6. Wearable identification software 340 may identify a specific sweat test strip and is described in more detail herein in the context of FIG. 12. Wearable software 336 may further comprise a wearable manual choice software 344, a wearable monitoring software 346, and a wearable action software 348, which may include a sweat analysis sensor data GUI 350 and a user alert GUI 352, as described further herein in the context of FIGS. 7A and 7B. Wearable manual choice software 344 may allow the user to begin a manual sweat test or to close user start GUI 342, which may prompt the execution of wearable monitoring software 346 that may monitor the sensor data to see if there is a match with one or more test triggering physiological conditions contained in wearable alert database 334. Wearable manual choice software 344 is discussed in detail below in the context of FIG. 13. Wearable monitoring software 346 is discussed in detail below in the context of FIG. 14A. Wearable action software 348 is discussed in greater detail below in the context of FIG. 14B.

It will be apparent that while various embodiments are described in terms of the wearable software 336, portions thereof, or other software or instructions “performing” various functionalities, such functionalities will in fact be performed by hardware components, such as a processor. As such, the wearable device 302 and server 304 may each include respective processors. As used herein, the term processor will be understood to encompass microprocessors, field programmable gate arrays (FPGAs), application-specific integrated circuits (ASICs), and other hardware devices capable of processing data in accordance with the described functionalities. In various embodiments wherein one or more ASICs are hard-wired to perform one or more of the functionalities described herein, software or instructions for defining such functionalities may be omitted. Further, as used herein, the term “memory” will be understood to encompass both volatile memories (e.g., L1/L2/L3 cache which may be implemented in SRAM, or system memory which may be implemented in DRAM) and nonvolatile memories (e.g., storage which may be implemented in flash, magnetic, or optical memories) but to exclude transitory signals per se.

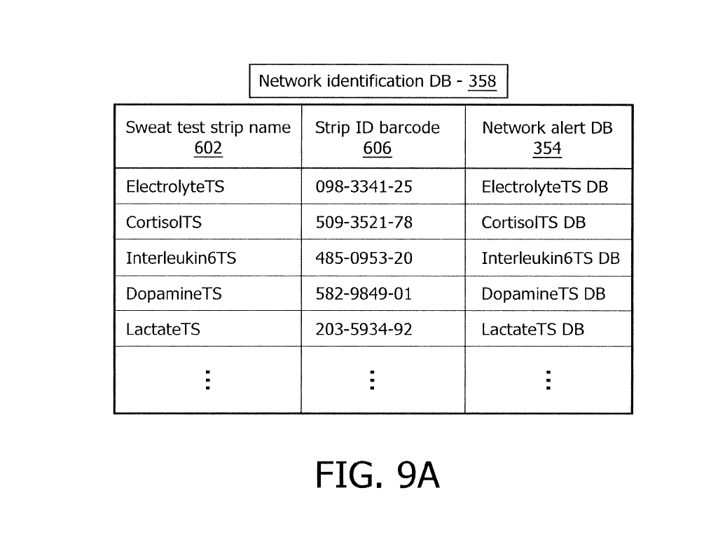

Still referring to FIG. 3, server 304 of WSA system 300 may include a server alert database 354, server identification software 356, a server identification database 358, and a server communications interface 360, shown here as “Server Comm.” Server alert database 354 may store a plurality of alert databases specific to a sweat-test type, such as an electrolyte sweat-test type. Server alert database 354 is discussed in detail below in the example of FIG. 8. Server communications interface 360 may be communicatively connected via the communication network 306 to wearable device 302. Server identification software 356 may receive strip identification barcode 312 data from wearable identification software 340 on wearable device 302 and may compare the strip identification barcode data to data in server identification database 358 to identify the sweat test strip 310 and, when the sweat test strip is identified, may provide information regarding the sweat test strip to the wearable identification software. It is noted that sweat test strip 310 may be configured for any one or more of a number of different sweat-test types. Exemplary sweat-test types may include, but not be limited to, sweat-test types that measure electrolyte levels, cortisol levels, interleukin six levels, dopamine levels, and lactate levels of a user.

In various embodiments, the wearable device 302 may not defer to the server 304 for the functions described herein. For example, in some embodiments, the wearable device 302 itself may include one or more of the components 354-360 for performing these functions locally. As another alternative, the wearable device 302 may instead communicate with a mobile device, tablet, personal computer, or other device of the user which may include one or more of the components 354-360 for performing these functions to serve the wearable device 302.

Referring again to FIG. 1, overall method 100 of FIG. 1 is described with reference to elements of WSA system 300 of FIG. 3 described above. At step 105, wearable device 302 receives an indication of a selected sweat-test type. This indication may be provided, for example, by a user manually entering the sweat-test type via user start GUI 342, by scanning strip ID barcode 312 with optical scanner 322, or by communicating via short range wireless communication (e.g., NFC or Bluetooth) with a chip attached to or otherwise associated with (e.g., on packaging of) the test strip. Identifying the sweat-test type may be performed via wearable identification software 340, server identification database 358, and server identification software 356. Server identification database 358 stores sweat-test type identification data that may be compared to user indicated sweat-test type data to match a sweat-test type. At step 110, determine which one or more of a plurality of physiological sensors to use for sensing one or more test-triggering physiological conditions. Step 110 may be performed by modules of wearable software 338. Test-triggering physiological conditions, test-trigger data, as well as other data stored in server alert database 354 are copied by server identification software 356 for a matching entry in server identification database 358 and sent to wearable identification software 340. Wearable identification software 340 may also download a test-specific algorithm from server 304, based on the selected sweat-test type, for determining which one or more of the plurality of physiological sensors to use based on the selected sweat-test type. At step 115, wearable device 302 collects sensor data using the one or more determined physiological sensors. There are two different methods for initiating collection of sensor data. A user, through interaction with user start GUI 342 may manually begin a sweat test or close the user start GUI 342 to initiate monitoring of sensor data until a test-triggering physiological condition is detected, at which point a user is instructed to begin a sweat test. Wearable manual choice software 344 monitors whether or not user start GUI 342 is closed or not and if a user selects to begin a sweat test manually or not. If it is determined by wearable manual choice software 344, that user start GUI 342 has been closed, then wearable monitoring software 346 is executed. If it is determined by wearable manual choice software 344 that a user has selected to begin a sweat test, then wearable action software 348 may be executed. Execution of either wearable monitoring software 346 or wearable action software 348 includes collection of sensor data using one or more determined physiological sensors.

Continuing through overall method 100 of FIG. 1, at step 120, it may be determined whether the one or more test-triggering physiological conditions are present in the user. Wearable monitoring software 346 may make this determination by comparing sensor data to test-trigger data to determine if one or more test-triggering physiological conditions are present. At step 125, the user may be instructed to deploy sweat test strip 310 at a location on the user and to initiate the sweat test in response to determining that at least one of the one or more test-triggering physiological conditions is present. Wearable action software 348, when executed, may display at user alert GUI 352 instructions for when and where to deploy sweat test strip 310. In an embodiment, user alert GUI 352 may also display the one or more test-triggering physiological conditions determined to be present in the user. Proceeding through overall method 100, at step 130, the user is provided instructions to end the sweat test. Wearable action software 348, when executed, may determine the instructions that may be displayed on user alert GUI 352. In an embodiment, instructions for processing results of a sweat test may also be displayed on user alert GUI 352.

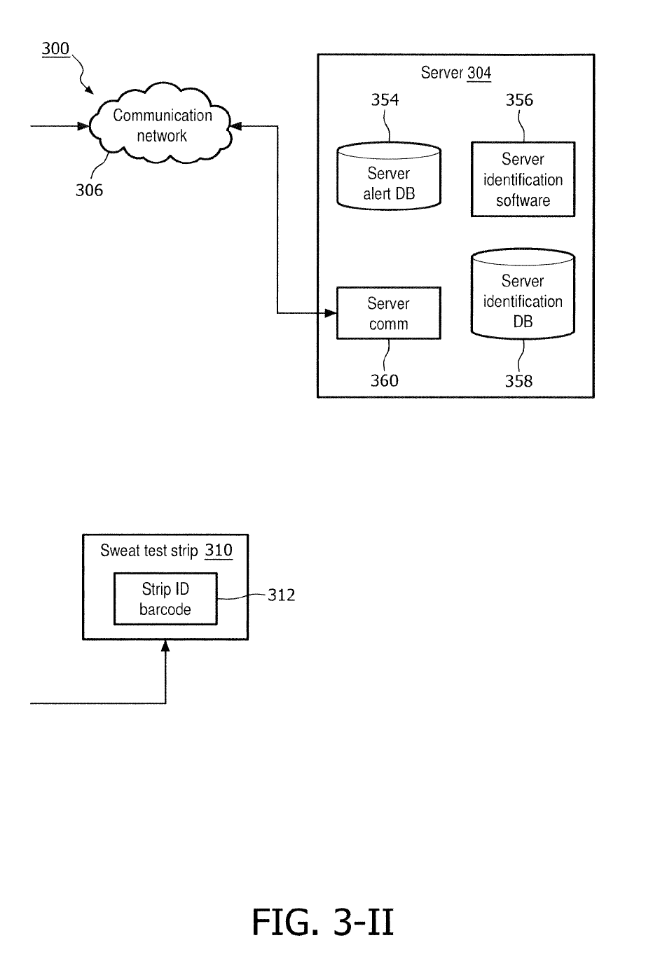

FIG. 4 is a block diagram of an exemplary wearable computing device 400 that may be configured to implement any one or more of various features and/or processes of the present disclosure, such as the features and processes illustrated in other figures of this disclosure, as well as features and processes that would be apparent to those of ordinary skill in the art after reading this entire disclosure. As shown, computing device 400 may include a memory interface 404, one or more data processors, image processors and/or central processing units 408, and a peripherals interface 412. Memory interface 404, one or more processors 408, and/or peripherals interface 412 may be separate components or may be integrated in one or more integrated circuits. The various components in computing device 400 may be coupled by one or more communication buses or signal lines.

Sensors, devices, and subsystems may be coupled to peripherals interface 412 to facilitate one or more functionalities. For example, a motion sensor 416, a light sensor 420, and a proximity sensor 424 may be coupled to peripherals interface 412 to facilitate orientation, lighting, and/or proximity functions. Other sensors 428 may also be connected to peripherals interface 412, such as a global navigation satellite system (GNSS) (e.g., GPS receiver), a temperature sensor, a biometric sensor, and/or one or more other sensing devices, to facilitate related functionalities.

A camera subsystem 432 and an optical sensor 436, e.g., a charged coupled device (CCD) or a complementary metal-oxide semiconductor (CMOS) optical sensor, may be utilized to facilitate camera functions, such as recording images and/or video. Camera subsystem 432 and optical sensor 436 may be used to collect images of a user to be used during authentication of a user, e.g., by performing facial recognition analysis.

Communication functions may be facilitated through one or more wireless communication subsystems 440, which may include radio frequency receivers and transmitters and/or optical (e.g., infrared) receivers and transmitters. The specific design and implementation of communication subsystem 440 may depend on the communication network(s) over which computing device 400 is intended to operate. For example, computing device 400 may include communication subsystems 440 designed to operate over a GSM network, a GPRS network, an EDGE network, a WI-FI™ or WiMax™ network, and/or a BLUETOOTH™ network. In particular, wireless communication subsystems 440 may include hosting protocols such that one or more devices 400 may be configured as a base station for other wireless devices.

An audio subsystem 444 may be coupled to a speaker 448 and a microphone 452 to facilitate voice-enabled functions, such as speaker recognition, voice replication, digital recording, and/or telephony functions. Audio subsystem 444 may be configured to facilitate processing voice commands, voice-printing, and voice authentication.

I/O subsystem 456 may include a touch-surface controller 460 and/or other input controller(s) 464. Touch-surface controller 460 may be coupled to a touch surface 468. Touch surface 468 and touch-surface controller 460 may, for example, detect contact and movement or a lack thereof using one or more of any of a plurality of touch sensitivity technologies, including but not limited to capacitive, resistive, infrared, and/or surface acoustic wave technologies, optionally as well as other proximity sensor arrays and/or other elements for determining one or more points of contact with touch surface 468.

Other input controller(s) 464 may be coupled to other input/control devices 472, such as one or more buttons, rocker switches, thumb-wheel, infrared port, USB port, and/or a pointer device such as a stylus. One or more related buttons or other controls (not shown) may include one or more sets of up/down buttons for volume and/or amplitude control of speaker 448 and/or microphone 452. Using the same or similar buttons or other controls, a user may activate a voice control, or voice command, module that enables the user to speak commands into microphone to cause device 400 to execute the spoken command. The user may customize functionality of one or more buttons or other controls. Touch surface 468 may, for example, also be used to implement virtual or soft buttons and/or a keyboard.

In some implementations, computing device 400 may present recorded audio and/or video files, such as MP3, AAC, and/or MPEG files. In some implementations, computing device 400 may include the functionality of an MP3 player, such as an iPod™. Computing device 400 may, therefore, include a 36-pin connector that is compatible with related iPod™ hardware. Other input/output and control devices may also be used.

As shown, memory interface 404 may be coupled to one or more types of memory 476. Memory 476 may include high-speed random access memory and/or non-volatile memory, such as one or more magnetic disk storage devices, one or more optical storage devices, and/or flash memory (e.g., NAND, NOR). Memory 476 may store an operating system 480, such as Darwin™ RTXC, LINUX, UNIX, OS X™, WINDOWS™, and/or an embedded operating system such as VxWorks. Operating system 480 may include instructions for handling basic system services and/or for performing hardware dependent tasks. In some implementations, operating system 480 may comprise a kernel (e.g., UNIX kernel). Further, in some implementations, operating system 480 may include instructions for performing voice authentication.

Memory 476 may also store communication instructions 482 to facilitate communicating with one or more additional devices, one or more computers, and/or one or more servers. Additionally or alternatively, memory 476 may include: graphical user interface instructions 484 to facilitate graphic user interface processing; sensor processing instructions 486 to facilitate sensor-related processing and functions; phone instructions 488 to facilitate phone-related processes and functions; electronic messaging instructions 490 to facilitate electronic-messaging related processes and functions; web browsing instructions 492 to facilitate web browsing-related processes and functions; media processing instructions 494 to facilitate media processing-related processes and functions; GNSS/Navigation instructions 496 to facilitate GNSS and navigation-related processes and instructions; and/or camera instructions 497 to facilitate camera-related processes and functions. Memory 476 may store other software instructions 498 to facilitate other processes and functions. For example, other software instructions 498 may include instructions for counting steps the user takes when device 400 is worn.

Memory 476 may also store other software instructions (not shown), such as web video instructions to facilitate web video-related processes and functions and/or web shopping instructions to facilitate web shopping-related processes and functions. In some implementations, media processing instructions 494 may be divided into audio processing instructions and video processing instructions to facilitate audio processing-related processes and functions and video processing-related processes and functions, respectively. An activation record and International Mobile Equipment Identity (IMEI) 499 or similar hardware identifier may also be stored in memory 476.

Each of the above identified instructions and applications may correspond to a set of instructions for performing one or more functions described herein. These instructions need not necessarily be implemented as separate software programs, procedures, or modules. Memory 476 may include additional instructions or fewer instructions. Further, various functions of computing device 400 may be implemented in hardware and/or in software, including in one or more signal processing and/or application specific integrated circuits.

FIG. 5A illustrates an exemplary embodiment of sweat test strip 310 of FIG. 3. As shown, sweat test strip 310 may include a sweat permeable adhesive layer 500, a cellophane layer 502, a sweat absorbing patch 504, a top protective textile layer 506, and strip identification barcode 312. Sweat permeable adhesive layer 500 may allow sweat test strip 310 to stick to the user's skin while simultaneously allowing sweat to permeate therethrough. Cellophane layer 502 may substantially prevent sweat from permeating through to sweat absorbing patch 504, as the cellophane layer itself is impermeable to sweat, and may have an integrated pull tab 510 to allow a user to remove the cellophane layer when the user desires to test their sweat. Once cellophane layer 502 is removed, sweat absorbing patch 504 may be able to absorb sweat. In some embodiments, sweat absorbing patch 504 may further include color change functionality. For example, sweat absorbing patch 504 may turn a particular color or shade when a user's sodium content is too high or too low. Top protective textile layer 506 may prevent any other moisture or contaminants from the surrounding environment from coming into contact with sweat absorbing patch 504 and may include strip ID barcode 312, which may enable optical scanner 322 (FIG. 3) to identify sweat test strip 310. FIG. 5B illustrates exemplary sweat test strip 310 applied to forearm 508 of a user, although it is emphasized that the sweat test strip may be fastened to any appropriate part of the user's body. As noted above, pull-tab 510 of cellophane layer 502 accommodates for quick and easy testing, as the user may simply pull on pull-tab 510 of cellophane layer 502 to initiate a sweat test in accordance with an embodiment of the current invention.

Referring now to FIG. 6, and also to FIG. 3, FIG. 6 illustrates an exemplary embodiment of user start GUI 342 of FIG. 3 to be displayed, for example, on a display device (which, in some embodiments, is coupled with a touchscreen input device) of the wearable device or of another device (e.g., a mobile phone, tablet, or pc) of the user configured to control the wearable device. Various alternative GUIs for performing the functionality described herein will be apparent. Those skilled in the art will readily appreciate the types of features common to wearable devices that can be implemented in user start GUI 342. Those skilled in the art will also understand the myriad of common user-interactive graphical soft controls and elements that can be used to receive inputs from a user, solicit responses from the user, and otherwise communicate relevant information to the user. Therefore, these controls and elements need not be described herein for those skilled in the art to understand how to make and use the claimed invention to the fullest scope claimed. In this embodiment, user start GUI 342 includes a “Scan Sweat Test Strip” button 600, which when actuated allows the user to use optical scanner 322 to scan strip identification barcode 312. Sweat identification barcode data may be sent to server 304, which may execute network identification software 356 to compare the data from scan strip identification barcode 312 with data from the network identification database 358, and then send related sweat test strip data to wearable device 302, such as the name of the sweat test strip to be displayed on user start GUI 342, for example. User start GUI 342 may then display sweat test strip name as displayed in window 602. As shown in FIG. 6, in this example, the sweat test strip name displayed in window 602 is “ElectrolyteTS”. User start GUI 342 includes an “Accept” button 604, which allows a user to confirm that the sweat test strip name displayed in window 602, is the sweat test strip they would like to use. User start GUI 342 may further include a “Strip ID Barcode” window 606. Through user interaction, a user manually enters a sweat test strip ID barcode using a soft keypad 608 or by scanning strip identification barcode 312 with optical scanner 322. Once strip identification barcode 312 has populated window 606, through user interaction with a “Send” button 610, wearable device 302 may send the barcode data to server 304, where network identification software 356 may compare it with appropriate data in network identification database 358. Server 304 may then return the sweat test strip name as displayed in window 602, shown here as “ElectrolyteTS”, for display on user start GUI 342. In this example, user start GUI 342 also includes a “Begin Sweat Test” button 612 for allowing a user to manually begin a sweat test and a “Close” button 614 for allowing the user to initiate wearable monitoring software 346.

With continuing occasional reference to FIG. 3, FIG. 7A illustrates an exemplary embodiment of user alert GUI 352 of FIG. 3. Those skilled in the art will readily appreciate the types of features common to wearable devices that can be implemented in user alert GUI 352. Those skilled in the art will also understand the myriad of common user-interactive graphical soft controls and elements that can be used to receive inputs from a user, solicit responses from the user, and otherwise communicate relevant information to the user. Therefore, these controls and elements need not be described herein for those skilled in the art to understand how to make and use the claimed invention to the fullest scope claimed. In this embodiment, user alert GUI 352 may include a display 702 for displaying instructions to take a sweat test. User alert GUI 352 may further include a “Triggering Data” window 704, which displays test-triggering data, such as “Blood Pressure 88 mmHG,” for example. User alert GUI 352 may further include a “Cellophane Layer Has Been Removed” button 706, that a user may select after they have physically removed cellophane layer 502 from sweat test strip 310. After engaging “cellophane layer has been removed” button 706, wearable device 302 receives data from sweat test strip 310 indicating that the sweat test strip is now absorbing and testing sweat. User alert GUI 352 may also include a “Sweat Analysis Sensor Data” button 708 and a “Close” button 710. If “Sweat Analysis Sensor Data” button 708 is selected, sweat analysis sensor data GUI 350 opens.

FIG. 7B illustrates an exemplary embodiment of sweat analysis sensor data GUI 350 of FIG. 3, which may include the date and time and sensor data collected by one or more of physiological sensors 308(1) to 308(N) identified for use in performing method 100 of FIG. 1. Those skilled in the art will understand the myriad of common user-interactive graphical soft controls and elements that can be used to receive inputs from a user, solicit responses from the user, and otherwise communicate relevant information to the user. Therefore, these controls and elements need not be described herein for those skilled in the art to understand how to make and use the claimed invention to the fullest scope claimed. In this embodiment, sweat analysis sensor data GUI 350 includes a date and a time as well as one or more sensor readings, which, as shown in FIG. 7B, may include pulse, blood pressure, sleep total in the past 24 hours, and body temperature readings, among others. As shown in FIG. 7B, sensor data displayed on sweat analysis sensor data GUI 350 in this example are taken at ten second intervals. Sweat analysis sensor data GUI 350 may include a scroll bar 712 to scroll through sensor data during or after a sweat analysis test. Sweat analysis sensor data GUI 350 may also include a “Close” button 714 that through user interaction closes the sweat analysis sensor data GUI.

FIG. 8 illustrates an exemplary embodiment of network alert database 354 of FIG. 3. Network alert database 354 includes one or more databases corresponding to a sweat-test type. For example, as shown in FIG. 8, a first database may be an ElectrolyteTS Database 800 including an ElectrolyteTS Alert Database 802. As shown, ElectrolyteTS Alert Database 802 includes wearable sensor types (physiological sensors), wearable sensor alert levels (test-trigger data), and alert actions and finish actions. Wearable sensor alert levels are referred to interchangeably herein with test-trigger data. Alert actions may be instructions to deploy sweat test strip 310 of FIG. 3. Finish actions may include instructions to end the sweat test or instructions for sweat test processing following a sweat test. Physiological sensor data collected is compared to data in network alert database 354 to determine whether one or more test-triggering physiological conditions are present in the user. For example, for a sensor capable of measuring pulse, if a user's pulse is above 120 beats per minute (bpm) for 30 minutes, then an alert action is displayed on user alert GUI 352 of FIG. 3. A corresponding finish action is displayed after a sweat test has completed on user alert GUI 352. Network alert database 354 may include period 804, which represents the length of time for a particular sweat test. For example, here period 804 is “120 seconds” for ElectrolyteTS Alert Database 802 indicating that a sweat test length may be 120 seconds. Network alert database 354 may include a scroll bar 806 for scrolling through the plurality of databases included in the network alert database. In some embodiments, network alert database 354 may contain test-specific algorithms (not shown) that, when executed, determine which one or more of the plurality of physiological sensors to use and compare the sensor data to the test-trigger data so as to determine whether or not the one or more test-triggering physiological conditions are present in the user.

FIG. 9A illustrates an exemplary embodiment of network identification database 358 of FIG. 3. Network identification database 358 may, for example, store three types of data including one or more sweat test strip names (displayed in window 602), strip identification barcodes 606, and related network alert databases 354. Network identification database 358 is used in identification of sweat-test type. FIG. 9B illustrates an exemplary embodiment of wearable alert database 334 of FIG. 3. Wearable alert database 334 may be a specific database found in network alert database 354, such as that of FIG. 8, for a sweat test type.

FIGS. 10A and 10B illustrate exemplary embodiments of wearable sensor database 332 of FIG. 3 and exemplary wearable sweat analysis sensor database 330 of FIG. 3, respectively. Wearable sensor database 332 and wearable sweat analysis sensor database 330 may store similar or identical data, as shown here. As shown, they may both contain date and time information, as well as one or more sensor data readings, such as pulse, blood pressure, sleep, sleep total in the past 24 hours, and body temperature. A distinguishing factor between these two databases is that wearable sensor database 332 stores sensor data collected during execution of wearable monitoring software 346, while wearable sweat analysis sensor database 330 stores sensor data collected during execution of wearable action software 348. In some embodiments, data stored in wearable sweat analysis sensor database 330 is displayed to a user via sweat analysis sensor data GUI 350 like that of FIGS. 3 and 7B.

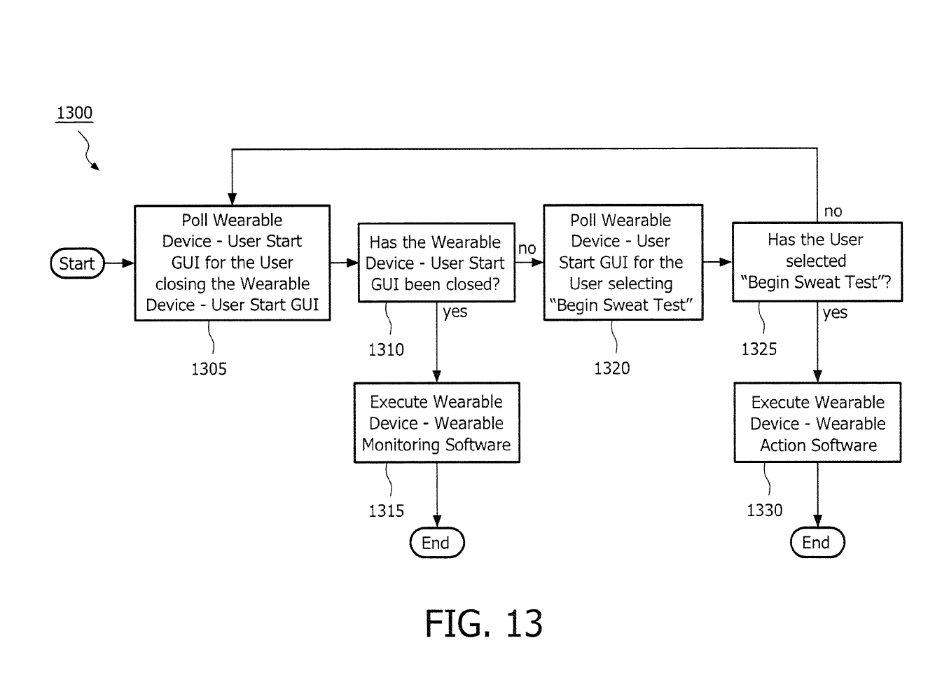

FIG. 11 illustrates an exemplary embodiment of wearable software method 1100 which may be executed, for example, by the wearable device 302 executing the wearable software 336. As shown, at step 1105, wearable identification software 340 is executed, and at step 1110, the wearable manual choice software 344 is executed after the strip ID barcode 312 has been sent to network identification software 356, and a matching sweat test strip name 602 and/or sweat-test type and network alert database 354 data have been sent back to wearable device 302. At step 1115, if the user has, for example, selected “Begin Sweat Test” button 612 on user start GUI 342, then wearable action software 348 is executed. If a user has not selected “Begin Sweat Test” button 612 on user start GUI 342, then at step 1120 user executes wearable monitoring software 346, which keeps collecting sensor data until one or more test-triggering physiological conditions are present in the user, as determined by comparison with test-trigger data. Once it is determined that one or more test-triggering physiological conditions are present in the user, at step 1125 user executes wearable action software 348, and the method ends.

FIG. 12 illustrates an exemplary embodiment of wearable identification software method 1200 (which may be performed by the wearable device 302 executing the wearable identification software 340) and a server identification software method 1202 (which may be performed by the server 304 executing the server identification software 356). Wearable identification software method 1200 and network identification software method 1202 work in parallel. Steps of wearable identification software method 1200 and network identification software method 1202 will be described herein together. At step 1205 of wearable identification software method 1200 user is allowed to identify strip identification barcode 312 of sweat test strip 310. For example, a user may either manually enter strip identification barcode 312 using keyboard 608, such as that of user start GUI 342 or initiate “Scan Sweat Test Strip” button 600 of the wearable device user start GUI 342, which allows the user to use optical scanner 322 to read strip identification barcode 312 on sweat test strip 310. Next, after strip identification barcode 312 data has been either scanned by optical scanner 322 or manually entered, related data is sent to network identification software 356 at step 1210. At step 1215, and now referring to network identification software method 1202, strip identification barcode 324 receives data from wearable identification software 340, and then, at step 1220, compares the strip identification barcode data with data stored in network identification database 358. Network identification database 358 may contain information regarding various sweat-test types, such as identifying barcode data and one or more network alert databases related to a particular sweat test type. At step 1225 of network identification software method 1202, if a match is found in network identification database 358 that matches the sweat test strip name identified in window 602, the method may proceed by sending that data to wearable identification software 340. At step 1230 of wearable identification software method 1200, wearable identification software 340 receives and displays the matching sweat test strip name on user start GUI 342. At step 1235 of wearable identification software method 1200, the user confirms that the matching sweat test strip name displayed on user start GUI 342 is for correct sweat test strip 310. This data is then sent back to network identification software 356. At step 1240 of network identification software method 1202, network identification software 356 receives a notification of the user confirmation from wearable identification software 340 and then retrieves and/or copies network alert database 354 specific to the matching entry (sweat-test type) in network identification database 358 at step 1245. Network alert database 358 may then be sent to wearable identification software 340. At step 1250 of wearable identification software method 1200, wearable identification software 340 may receive network alert database 358 specific to that sweat-test type, save the specific network alert database to wearable alert database 334, and then may execute wearable device wearable manual choice software 344, which ends methods 1200, 1202, respectively.

FIG. 13 illustrates an exemplary embodiment of wearable manual choice software method 1300 (which may be performed by the wearable device 302 executing the wearable manual choice software 344). As shown, at step 1305 of wearable manual choice software method 1300 wearable device 302 user start GUI 342 is polled to determine whether the user has closed the user start GUI (step 1310). If the user did close user start GUI 342, then wearable manual choice software 344 may execute wearable monitoring software 346 at step 1315 to start collecting sensor data and compare sensor data with wearable alert database 334 to determine whether or not one or more test-triggering physiological conditions are present in the user. If one or more physiological condition are present in the user, wearable action software 348 may be executed. If the user has not closed user start GUI 342, then at step 1320 of wearable manual choice software method 1300, wearable device 302 user start GUI 342 is polled to detect, at step 1325, if user selected a “Begin Sweat Test” button 612. If the user has not selected to manually begin the sweat test, then wearable manual choice software method 1300 loops back to step 1305 of polling user start GUI 342 to determine whether the user has closed the user start GUI. If a user has selected to manually begin a sweat test, then at step 1330 of wearable manual choice software method 1300, wearable action software 348 is executed, and the method ends.

FIG. 14A illustrates an exemplary embodiment of wearable monitoring software method 1400 (which may be performed by the wearable device 302 executing the wearable monitoring software 346). At step 1405 of wearable monitoring software method 1400, sensor and wearable clock data is collected. At step 1410, that data may be saved to wearable sensor database 332. At step 1415, the most recent sensor data entry from wearable sensor database 332 is retrieved, and then at step 1420, that most recent data entry from the wearable sensor database is compared with wearable alert database 334 to determine whether there is a match (step 1425). If there is a match, at step 1430, wearable action software 348 is executed. If there is no match, wearable monitoring software method 1400 loops back to step 1405. In summary, wearable monitoring software method 1400 may collect sensor data and compare the most recent sensor data with one or more test-triggers to determine whether one or more test-triggering physiological conditions are present in the user.

FIG. 14B illustrates an exemplary embodiment of wearable action software method 1435 (which may be performed by the wearable device 302 executing the wearable action software 348). As shown, at step 1440 of wearable action software method 1435, user alert GUI 342 is displayed, and at step 1445, the appropriate alert action for the matching data entry stored in wearable alert database 334 is executed. An example of an alert action is information and instruction that may be sent to user alert GUI 352 to provide an indication to a user when, e.g., their temperature is too high and/or to take a sweat test. An indication may include a message and related instructions. For example, the message and related instructions may include that the user is running a fever and that the user should test for interleukin 6 to test for a possible infection. At step 1450, user places sweat test strip 310 as instructed by user alert GUI 352 and selects “Cellophane Layer Has Been Removed” button 706 of the user alert GUI once the user has removed the cellophane layer from sweat test strip 310. At step 1455, user starts wearable timer 316, and then, at step 1460, data from sensors and wearable clock 312 is collected via wearable action software 348. At step 1465, data is saved to wearable sweat analysis sensor database 330, and then sent and displayed on sweat analysis sensor data GUI 350. Through user interaction with sweat analysis sensor data GUI 350, a user may review sensor data as well as wearable device 302 data. At step 1470, if wearable timer 316 has reached a time equal to the period that was set for a sweat-test type, then at step 1475 an appropriate finish action for the matching data entry in wearable alert database 336 is executed. If the period of time as defined in wearable alert database 334 has not been reached at step 1470, then wearable action software method 1435 loops back to step 1460.

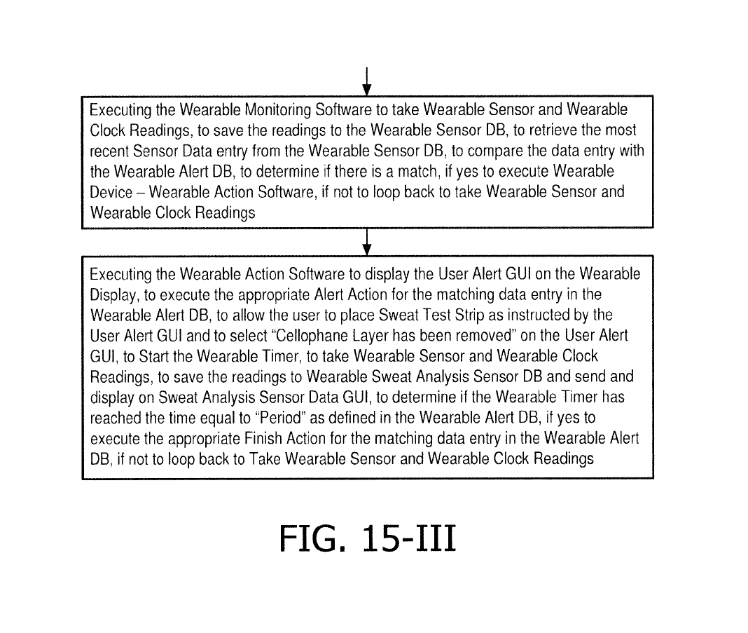

FIG. 15 illustrates an exemplary embodiment of a method 1500 of providing sweat health data to a user. Steps of method 1500 for providing sweat health data to a user are self-explanatory given the disclosure above. It is noted that this particular method is provided only for example and that the steps of method 1500 may occur in a different order and some steps may be omitted.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

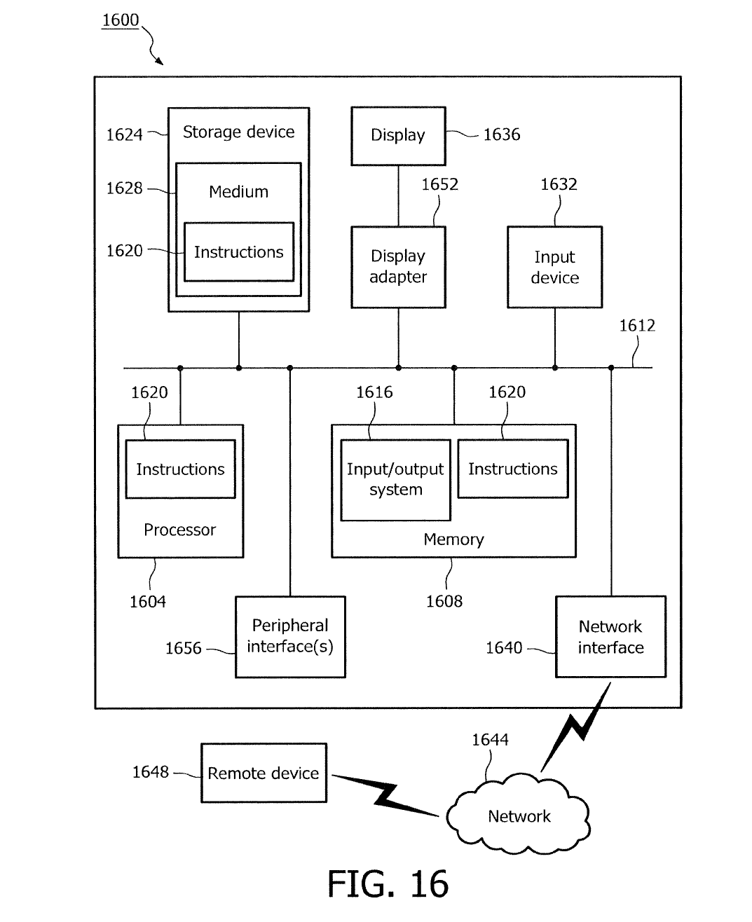

FIG. 16 shows a diagrammatic representation of one embodiment of a computing device in the exemplary form of a computer system 1600 within which a set of instructions for causing a control system, such as the WSA system 300 of FIG. 3, to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1600 includes a processor 1604 and a memory 1608 that communicate with each other, and with other components, via a bus 1612. Bus 1612 may include any of several types of bus structures including, but not limited to, a memory bus, a memory controller, a peripheral bus, a local bus, and any combinations thereof, using any of a variety of bus architectures.

Memory 1608 may include various components (e.g., machine-readable media) including, but not limited to, a random access memory component, a read only component, and any combinations thereof. In one example, a basic input/output system 1616 (BIOS), including basic routines that help to transfer information between elements within computer system 1600, such as during start-up, may be stored in memory 1608. Memory 1608 may also include (e.g., stored on one or more machine-readable media) instructions (e.g., software) 1620 embodying any one or more of the aspects and/or methodologies of the present disclosure. In another example, memory 1608 may further include any number of program modules including, but not limited to, an operating system, one or more application programs, other program modules, program data, and any combinations thereof.

Computer system 1600 may also include a storage device 1624. Examples of a storage device (e.g., storage device 1624) include, but are not limited to, a hard disk drive, a magnetic disk drive, an optical disc drive in combination with an optical medium, a solid-state memory device, and any combinations thereof. Storage device 1624 may be connected to bus 1612 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 1624 (or one or more components thereof) may be removably interfaced with computer system 1600 (e.g., via an external port connector (not shown)). Particularly, storage device 1624 and an associated machine-readable medium 1628 may provide nonvolatile and/or volatile storage of machine-readable instructions, data structures, program modules, and/or other data for computer system 1600. In one example, software 1620 may reside, completely or partially, within machine-readable medium 1628. In another example, software 1620 may reside, completely or partially, within processor 1604.

Computer system 1600 may also include an input device 1632. In one example, a user of computer system 1600 may enter commands and/or other information into computer system 1600 via input device 1632. Examples of an input device 1632 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1632 may be interfaced to bus 1612 via any of a variety of interfaces (not shown) including, but not limited to, a serial interface, a parallel interface, a game port, a USB interface, a FIREWIRE interface, a direct interface to bus 1612, and any combinations thereof. Input device 1632 may include a touch screen interface that may be a part of or separate from display 1636, discussed further below. Input device 1632 may be utilized as a user selection device for selecting one or more graphical representations in a graphical interface as described above.

A user may also input commands and/or other information to computer system 1600 via storage device 1624 (e.g., a removable disk drive, a flash drive, etc.) and/or network interface device 1640. A network interface device, such as network interface device 1640, may be utilized for connecting computer system 1600 to one or more of a variety of networks, such as network 1644, and one or more remote devices 1648 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1644, may employ a wired and/or a wireless mode of communication. In general, any network topology may be used. Information (e.g., data, software 1620, etc.) may be communicated to and/or from computer system 1600 via network interface device 1640.

Computer system 1600 may further include a video display adapter 1652 for communicating a displayable image to a display device, such as display device 1636. Examples of a display device include, but are not limited to, a liquid crystal display (LCD), a cathode ray tube (CRT), a plasma display, a light emitting diode (LED) display, and any combinations thereof. Display adapter 1652 and display device 1636 may be utilized in combination with processor 1604 to provide graphical representations of aspects of the present disclosure. In addition to a display device, computer system 1600 may include one or more other peripheral output devices including, but not limited to, an audio speaker, a printer, and any combinations thereof. Such peripheral output devices may be connected to bus 1612 via a peripheral interface 1656. Examples of a peripheral interface include, but are not limited to, a serial port, a USB connection, a FIREWIRE connection, a parallel connection, and any combinations thereof.

It should be apparent from the foregoing description that various example embodiments of the invention may be implemented in hardware or firmware. Furthermore, various exemplary embodiments may be implemented as instructions stored on a machine-readable storage medium, which may be read and executed by at least one processor to perform the operations described in detail herein. A machine-readable storage medium may include any mechanism for storing information in a form readable by a machine, such as a personal or laptop computer, a server, or other computing device. Thus, a machine-readable storage medium may include read-only memory (ROM), random-access memory (RAM), magnetic disk storage media, optical storage media, flash-memory devices, and similar storage media.

It should be appreciated by those skilled in the art that any block diagrams herein represent conceptual views of illustrative circuitry embodying the principles of the invention. Similarly, it will be appreciated that any flow charts, flow diagrams, state transition diagrams, pseudo code, and the like represent various processes which may be substantially represented in machine readable media and so executed by a computer or processor, whether or not such computer or processor is explicitly shown.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

1. A computer implemented method of assisting a user with administering a sweat test using a sweat test strip and a wearable device comprising a plurality of physiological sensors, the method comprising:
receiving, via the wearable device, an indication of a selected sweat-test type from the user;
determining, on the wearable device and based on the selected sweat-test type, which one or more of the plurality of physiological sensors to use for sensing one or more test-triggering physiological conditions of the user;
collecting, on the wearable device, sensor data using the determined one or more of the plurality of physiological sensors;
determining, on the wearable device using the sensor data, whether or not the one or more test-triggering physiological conditions are present in the user;
in response to determining that at least one of the one or more test-triggering physiological conditions is present, instructing, by the wearable device, the user to deploy the sweat test strip to a location on the user and to initiate the sweat test; and
providing instruction, by the wearable device, to the user for interpretation of the sweat test strip.

2. A method according to claim 1, further comprising:
downloading, by the wearable device based on the selected sweat-test type, a test-specific algorithm;
downloading, by the wearable device, test-trigger data; and
executing, on the wearable device, the test-specific algorithm, wherein said executing the test-specific algorithm includes:
determining, on the wearable device and based on the selected sweat-test type, which one or more of the plurality of physiological sensors to use; and
comparing the sensor data to the test-trigger data so as to determine whether or not the one or more test-triggering physiological conditions are present in the user.

3. A method according to claim 1, wherein said collecting sensor data includes collecting sensor data from at least one of the determined physiological sensors located onboard the wearable device.

4. A method according to claim 1, wherein said collecting sensor data includes collecting sensor data from at least one physiological sensor in contact with the user.

5. A method according to claim 1, wherein said instructing the user to deploy the sweat test strip to a location on the user and to initiate the sweat test includes displaying instructions on a user alert graphical user interface located onboard the wearable device.

6. A method according to claim 5, wherein said displaying the instructions further includes displaying, by the user alert graphical user interface, the one or more test-triggering physiological conditions present in the user.

7. A method according to claim 1, wherein said instructing the user to end the sweat test includes displaying instructions on a user alert graphical user interface located onboard the wearable device.

8. A method according to claim 7, wherein said displaying the instructions further includes displaying, by the user alert graphical user interface, instructions for processing results of a sweat test.

9. A method according to claim 1, wherein said receiving an indication of a selected sweat-test type from the user includes scanning a sweat test strip with an optical scanner connected to the wearable device.

10. A method according to claim 9, wherein the sweat test strip includes a strip identification barcode and said scanning includes scanning the strip identification barcode.

11. A machine-readable storage medium containing machine-executable instructions for assisting a user with administering a sweat test using a sweat test strip and a wearable device comprising a plurality of physiological sensors, said machine-executable instructions comprising:
a first set of machine-executable instructions for receiving, via the wearable device, an indication of a selected sweat-test type from the user;
a second set of machine-executable instructions for determining, on the wearable device and based on the selected sweat-test type, which one or more of the plurality of physiological sensors to use for sensing one or more test-triggering physiological conditions of the user;
a third set of machine-executable instructions for collecting, on the wearable device, sensor data using the determined one or more of the plurality of physiological sensors;
a fourth set of machine-executable instructions for determining, on the wearable device using the sensor data, whether or not the one or more test-triggering physiological conditions are present in the user;
a fifth set of machine-executable instructions for, in response to determining that at least one of the one or more test-triggering physiological conditions is present, instructing, by the wearable device, the user to deploy the sweat test strip to a location on the user and to initiate the sweat test; and
a sixth set of machine-executable instructions for instructing, by the wearable device, the user to interpret the sweat test strip.

12. A machine-readable storage medium according to claim 11, wherein said fourth set of machine-executable instructions includes machine-executable instructions for:
downloading, by the wearable device based on the selected sweat-test type, a test-specific algorithm;
downloading, by the wearable device, test-trigger data; and
executing, on the wearable device, the test-specific algorithm, wherein said executing the test-specific algorithm includes:
determining, on the wearable device and based on the selected sweat-test type, which one or more of the plurality of physiological sensors to use; and
comparing the sensor data to the test-trigger data so as to determine whether or not the one or more test-triggering physiological conditions are present in the user.

13. A machine-readable storage medium according to claim 11, wherein said third set of machine-executable instructions includes machine-executable instructions, for collecting sensor data from at least one of the determined physiological sensors located onboard the wearable device.

14. A machine-readable storage medium according to claim 11, wherein said second set of machine-executable instructions includes machine-executable instructions, for collecting sensor data from at least one physiological sensor in contact with the user.

15. A machine-readable storage medium according to claim 11, wherein said fifth set of machine-executable instructions includes machine-executable instructions, for displaying instructions on a user alert graphical user interface located onboard the wearable device.

16. A system comprising:
a wearable device with a plurality of integrated physiological sensors; and
a compartment in the wearable device that stores a sweat test strip;
wherein the wearable device
receives an indication of a selected sweat-test type;
determines, based on the selected sweat-test type, which one or more of the plurality of physiological sensors to use for sensing one or more test-triggering physiological conditions of a wearer of the wearable device;
collects sensor data using the determined one or more of the plurality of physiological sensors;
determines, using the sensor data, whether or not the one or more test-triggering physiological conditions are present in the wearer;
instructs, in response to determining that at least one of the one or more test-triggering physiological conditions is present, the wearer to deploy the sweat test strip from the compartment to a slot in the wearable device for holding the sweat test strip against a location on the wearer to initiate the sweat test; and
provides instruction to the wearer for interpretation of the sweat test strip.
